(19) United States
(12) Patent Application Publication
(10) Pub. No.: US 2015/0374286 A1
(43) Pub. Date: Dec. 31, 2015

(54) DEVICE AND METHOD FOR RESPIRATORY EXERCISE

(71) Applicant: HOSPITAL SANT JOAN DE DEU, Esplugues de Llobregat (Barcelona) (ES)

(72) Inventors: Natalia RODRÍGUEZ NIEVA, Esplugues de Llobregat (Barcelona) (ES); Jordi CABRE MARTÍNEZ, Espluges de Llobregat (Barcelona) (ES); Enrique DEL CAMPO GARCÍA RAMOS, Esplugues de Llobregat (Barcelona) (ES); Anna FEBRER ROTGER, Esplugues de Llobregat (Barcelona) (ES); Angel ALONSO MERINO, Esplugues de Llobregat (Barcelona) (ES)

(21) Appl. No.: 14/768,343

(22) PCT Filed: Feb. 20, 2014
(86) PCT No.: PCT/ES2014/070126

§ 371 Date: Aug. 17, 2015

(30) Foreign Application Priority Data
Feb. 20, 2013 (ES) P201330226

(51) Int. Cl. A61B 5/00; A61M 16/20; A61B 5/09; A61B 7/04; A61B 7/00; A61M 16/00

(52) U.S. Cl. CPC A61B 5/4833; A61B 7/003; A61M 16/0003; A61B 5/09; A61B 7/04; A61M 16/201; A61B 5/0004; A61B 5/742; A61M 2205/59; A61B 2503/06

(57) ABSTRACT

The respiratory exercise device comprises means for detecting the respiration of a person and it is characterized in that said means for detecting the respiration of a person comprises a turbine whistle (3) which generates a variable frequency sound as a function of the air flow which passes through the same and a microphone (4) which captures said variable frequency sound generated by the turbine whistle (3).

The method for respiratory exercise comprises the steps of obtaining data relating to the respiration of a person with the previous device, sending said data relating to the respiration to control means; and applying an algorithm to said data for calculating the values of the flows generated by the respiration.

It allows respiratory exercise to be carried out in a simple manner, even for children.

### BACKGROUND OF THE INVENTION

Respiratory physiotherapy is indicated as a coadjuvant treatment for many respiratory diseases with the following objectives: maintain the airways free from of secretions, achieve good mobility of the thoracic cavity and maintain good functioning of the respiratory muscles.

Respiratory processes, which are present with an increase of secretions, can cause bronchial obstruction if they are not mobilized with subsequent bacterial superinfection and possible pulmonary involvement.

Respiratory physiotherapy is composed of a series of respiratory exercises which achieve the objective of cleaning the airway, providing the arrival of air to all the possible spaces, mobilizing and expelling the secretions which had become stuck and facilitating such that the air can enter and exit from the airway without any problems. All of which is going to definitively lead to preventing or stopping a possible deterioration of the pulmonary function.

One of the principal problems which conventional respiratory physiotherapy for children poses is its difficulty in creating adherence in the form of a habit, due to the fact that the child ends up becoming tired of always practicing the same exercises.

Therefore, the need for a device is evident which allows for a child to utilize it as a game or for an adult to comfortably utilize it, the air flow being able to be readily detected and the need for a method which allows suitable monitoring.

In addition, devices are known which comprise a microphone which detect respiratory sounds during sleep, which are subsequently analyzed in order to detect possible sleep disruptions. However, these currently known devices are only geared to detect sleep disruptions, but not to be used as respiratory exercise, in particular in children.

### DESCRIPTION OF THE INVENTION

Using the device and the method of the invention, the drawbacks cited can be solved, presenting other advantages which are described below.

According to a first aspect, the invention relates to a device for respiratory exercise, which comprises means for detecting the respiration of a person and is characterized in that said means for detecting the respiration of a person comprise a turbine whistle which generates a variable frequency sound as a function of the air flow which passes through the same, and a microphone which captures said variable frequency sound generated by the turbine whistle.

Advantageously the respiratory exercise device of the present invention also comprises a filter associated with said microphone.

According to a preferred embodiment, the respiratory exercise device also comprises an adjustable valve inserted at one air inlet end.

Furthermore, advantageously, the said microphone comprises means for sending the data of the captured sound generated by the turbine whistle, said means being, for example an antenna, wireless means or a connector.

The respiratory exercise device of the present invention preferably comprises an elongated hollow body, the microphone being located outside of said body and said turbine whistle being located in the interior of said body.

According to a second aspect, the present invention also relates to a method for respiratory exercise, which is characterized in that it comprises the steps of:
obtaining data relating to the respiration of a person with the previously indicated device;
sending said data relating to the respiration to a computer; and
applying an algorithm to said data to calculate the values of the flows generated by the respiration.

Advantageously the method for respiratory exercise also comprises the step of storing the values of the flow generated by the respiration for the monitoring thereof.

Preferably the method for respiratory exercise of the present invention also comprises the step of converting the values of the flows generated by the respiration into real-time moving images, for example into a video game suitable for respiratory exercise in children or in adults.

According to a preferred embodiment, in the application of said algorithm the zero-cross rate (ZCR) is calculated and a zero-cross rate conversion table is applied to the flow.

Using the device and the method of the present invention, respiratory exercise is achieved in a simple manner, even for children, using a simple device and a method which allows the monitoring on the part of a doctor and furthermore allows the respiratory exercise to be converted into a video game which incentivizes the use thereof in children and in adults.

### BRIEF DESCRIPTION OF THE DRAWINGS

For a better understanding of what has been stated, drawings are enclosed, in which, schematically and only as a non-limiting example, a practical exemplary embodiment is depicted:

FIG. 1 is a sectional schematic lateral view of the device of the present invention according to a first embodiment; and

FIG. 2 is a sectional schematic lateral view of the device of the present invention according to a second embodiment.

### DESCRIPTION OF A PREFERRED EMBODIMENT

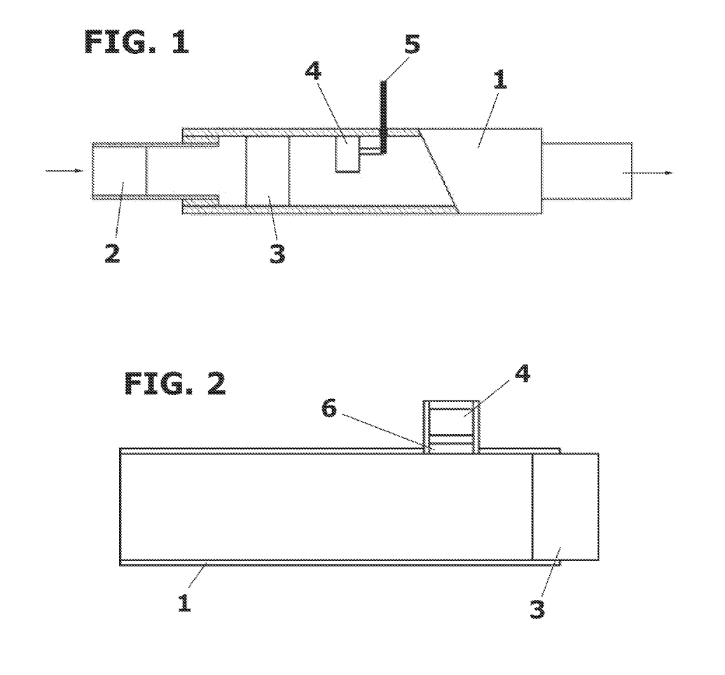

The respiratory exercise device of the present invention is formed by an elongated hollow body 1 which defines an air inlet end and an air outlet end and which comprises an adjustable valve 2 located at the air inlet end, a turbine whistle 3 and a microphone 4 as can be observed in FIG. 1, in a first embodiment.

A turbine whistle 3 is a whistle which generates a variable frequency sound as a function of the air flow which passes through the same such that the greater the flow that passes through, the greater the frequency. Furthermore, the device of the present invention also comprises connection means for sending the data of the variable frequency sound generated by the turbine whistle 3. For example, said means are an antenna 5 connected to said microphone 4 which sends the data relating to the sound detected, such as the frequency, to a computer (not shown) or any suitable platform. In order to not cause turbulences, said microphone is associated with a filter 6.

It should be indicated that said connection means can also be any suitable means, such as wireless means (Wi-Fi, Bluetooth®, etc.) or any type of connector (mini-jack, USB, etc.).

In this way, when the user blows through the air inlet end, the air flow causes the whistle turbine 3 to generate a variable frequency sound which is captured by the microphone 4 which sends the data relating to said frequency to a computer or any suitable platform.

In FIG. 2 a second embodiment of the device of the present invention is depicted. It should be indicated that for reasons of simplicity, in FIG. 2, only the elements of the device inserted differently with respect to the first embodiment are shown.

In particular, the principal difference is the arrangement of the microphone 4 outside of said elongated hollow body 1 to prevent turbulences affecting said microphone.

According to a second aspect, the present invention also relates to a respiratory exercise method which comprises the following steps:
when a user inhales or exhales into the previously indicated device, data relating to the respiration is generated, in particular frequency values;
said device sends the frequency values to control means, such as a computer;
in said control means, said frequency values pass through a conversion algorithm in which the zero-cross rate (ZCR) is detected and a zero-cross rate conversion table is applied to the flow, preferably in l/m; and
said information is stored for the subsequent monitoring thereof on the part of a doctor.

Furthermore, said method can also comprise an additional step of converting said information regarding the respiration flow into real-time moving images, for example a video game such that a child or an adult can play at the same time as carrying out the suitable respiratory exercises.

Even though reference is made to a specific embodiment of the invention, it is evident for a person skilled in the art that the device and the method described can have numerous variations and modifications and all the details mentioned can be substituted for other technical equivalents without extending beyond the scope of protection defined by the attached claims.

1. A respiratory exercise device comprising means for detecting the respiration of a person, wherein said means for detecting the respiration of a person comprises a turbine whistle which generates a variable frequency sound as a function of the air flow which passes through the same and a microphone which captures said variable frequency sound generated by the turbine whistle.

2. The respiratory exercise device according to claim 1, wherein it also comprises a filter associated with said microphone.

3. The respiratory exercise device according to claim 1, wherein it also comprises an adjustable valve inserted at an air inlet end of the device.

4. The respiratory exercise device according to claim 1, wherein it comprises an elongated hollow body, said microphone being located outside of said body and said turbine whistle being located in the interior of said body.

5. The respiratory exercise device according to claim 1, wherein said microphone comprises connection means for sending the data of the variable frequency sound generated by the turbine whistle.

6. The respiratory exercise device according to claim 5, wherein said means for sending the data of the variable frequency sound generated by the turbine whistle comprises an antenna, a wireless connection or a connector.

7. A method for respiratory exercise, comprising the steps of:
obtaining data relating to the respiration of a person with the device according to claim 1;
sending said data relating to the respiration to control means; and
applying an algorithm to said data for calculating the values of the flows generated by the respiration.

8. The method for respiratory exercise according to claim 7, wherein it also comprises the step of storing the values of the flows generated by the respiration for the monitoring thereof.

9. The method for respiratory exercise according to claim 7, wherein it also comprises the step of converting the values of the flows generated by the respiration into real-time moving images.

10. The method for respiratory exercise according to claim 7, wherein in the application of said algorithm the zero-cross rate is calculated and a zero-cross rate conversion table is applied to the flow.
